(19) United States
(12) Patent Application Publication
(10) Pub. No.: US 2024/0161926 A1
(43) Pub. Date: May 16, 2024

(54) PROGNOSTIC METHOD USING SEX SPECIFIC FEATURES OF TUMOR INFILTRATING LYMPHOCYTES

(71) Applicants: Case Western Reserve University, Cleveland, OH (US); Wisconsin Alumni Research Foundation, Madison, WI (US); Emory University, Atlanta, GA (US)

(72) Inventors: Olivia Krebs, Cleveland Heights, OH (US); Pallavi Tiwari, Waunakee, WI (US); Germán Corredor, Beachwood, OH (US)

(21) Appl. No.: 18/493,862

(22) Filed: Oct. 25, 2023

Related U.S. Application Data
(60) Provisional application No. 63/424,651, filed on Nov. 11, 2022.

(51) Int. Cl. G16H 50/20; G16H 30/00

(52) U.S. Cl. CPC G16H 50/20; G16H 30/00

(57) ABSTRACT

The present disclosure relates to a method. The method includes accessing a segmented digitized pathology image corresponding to a high grade glioma (HGG) patient having a first biological sex. The segmented digitized pathology image identifies nuclei. The nuclei are classified as tumor infiltrating lymphocytes (TILs) or non-TILs. Sex specific features related to the nuclei identified as the TILs and the non-TILs are extracted. The sex specific features characterize a spatial organization of the TILs and the non-TILs. The sex specific features are operated on with a sex specific machine learning model corresponding to the first biological sex to generate a sex specific patient risk score.

### REFERENCE TO RELATED APPLICATION

This application claims the benefit of U.S. Provisional Application No. 63/424,651, filed on Nov. 11, 2022, the contents of which are hereby incorporated by reference in their entirety.

### FEDERAL FUNDING NOTICE

This invention was made with government support under CA264017 awarded by the National Institutes of Health, W81XWH-21-1-0160 awarded by the Department of Defense, and 1937968 awarded by the National Science Foundation. The government has certain rights in the invention.

### BACKGROUND

High grade gliomas are an aggressive form of cancer that occurs in a patient's brain and/or spinal cord. The complexity of high grade gliomas and variability amongst patients make treatment of the disease very difficult. Furthermore, while treatments may slow progression of the cancer and reduce signs and symptoms, a cure is often not possible.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of a block diagram of a high grade glioma (HGG) assessment system comprising sex specific machine learning models configured to determine a sex specific patient risk score from a digitized pathology image.

FIG. 2 illustrates some embodiments of a flow diagram showing a method for using a sex specific machine learning model to determine a sex specific patient risk score for a HGG patient.

FIG. 3 illustrates some additional embodiments of a block diagram of a HGG assessment system comprising sex specific machine learning models configured to determine a sex specific patient risk score.

FIG. 4A illustrates some embodiments of a workflow for generating sex specific features from a digitized pathology image using graphs and nuclei clusters.

FIG. 4B illustrates some embodiments of a table showing exemplary sex specific spatial features extracted from graphs and nuclei clusters.

FIGS. 5A-5B illustrate some embodiments of digitized images showing visualizations of exemplary sex specific features.

FIG. 6 illustrates some additional embodiments of a block diagram of a HGG assessment system comprising sex specific machine learning models configured to determine a sex specific patient risk score.

FIG. 7 illustrates a table showing exemplary results of a Cox Regression model for different sex specific machine learning models according to a disclosed method and/or apparatus.

FIGS. 8A-8C illustrate some embodiments of Kaplan Meier survival curves corresponding to different sex specific machine learning models for determining overall survival for subgroups of female patients having HGG.

FIG. 9 illustrates a block diagram of some additional embodiments of a HGG assessment system comprising sex specific machine learning models that are configured to be trained to determine a sex specific patient risk score using sex specific features extracted from digitized pathology images.

FIG. 10 illustrates some embodiments of a flow diagram showing a method for training a sex specific machine learning model to determine a sex specific patient risk score using sex specific features extracted from digitized pathology images.

FIG. 11 illustrates some embodiments of a block diagram showing a workflow of the disclosed method for training sex specific machine learning models to determine a sex specific patient risk score.

FIGS. 12A-12C illustrate some embodiments of Kaplan-Meier survival curves determined based upon the disclosed method and corresponding to imaging data set associated with different biological sexes.

FIG. 13 illustrates a block diagram of some embodiments of a prognostic apparatus comprising a disclosed HGG assessment tool.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

High grade gliomas (HGGs) are highly aggressive tumors that begin within a patient's brain and/or spinal cord. Treatment for HGG typically includes multi-modal treatment that may start with surgery, followed by radiation and/or chemotherapy. Despite advances in the understanding of the disease and efforts to improve treatment outcomes, the prognosis for patients with HGG remains poor. For example, despite multi-modal treatment including surgical resection followed by radiotherapy with concomitant and adjuvant chemotherapy, glioblastoma which comprise the majority of HGGs, has a median survival of between 15 and 18 months.

Selecting a treatment option for a HGG patient presents a challenge for health care professionals due to the aggressive nature of the disease and the variance in efficacy of standard treatments. To improve patient treatments, there have been efforts to identify sub-groups of HGG patients that are likely to have favorable outcomes to certain treatments (e.g., to identify a sub-group of patients that respond well to immunotherapy). Unfortunately, so far none of these efforts have been successful. Hence, additional prognostic markers may be desirable to enable stratification of HGG disease risk for the purpose of informing individual treatment options.

It has been appreciated that computationally derived features related to the spatial organization of tumor-infiltrating lymphocytes (TILs) may be representative of an immune contexture within a tumor microenvironment. It has also been appreciated that there are immunological variances between people of different biological sexes. Because of such immunological variances, the spatial profiling of TILs may have prognostic value in determining overall survival for cancer patients of different biological sexes. Therefore, the use of computationally derived features relating to the spatial organization of TILs in machine learning models may be able to improve a prognostic value of such models.

Accordingly, the present disclosure relates to a method of determining a sex specific patient risk score for a HGG patient using a sex specific machine learning model that is configured to operate on sex specific features (e.g., sex specific spatial features) of TILs extracted from a digitized pathology image. In some embodiments, the method may comprise accessing a digitized pathology image of a HGG patient having a first biological sex, which has been segmented to identify nuclei. The nuclei are classified as tumor infiltrating lymphocytes (TILs) or non-TILs. Sex specific features relating to the spatial organization of the TILs and/or non-TILs are subsequently extracted. A sex specific machine learning model corresponding to the first biological sex is operated upon the sex specific features to generate a sex specific patient risk score. Operating the sex specific machine learning model upon the sex specific features allows for the sex specific machine learning model to account for immunological variances between different biological sexes, so as to enable the sex specific patient risk score to provide an accurate prognosis of overall survival in the HGG patient.

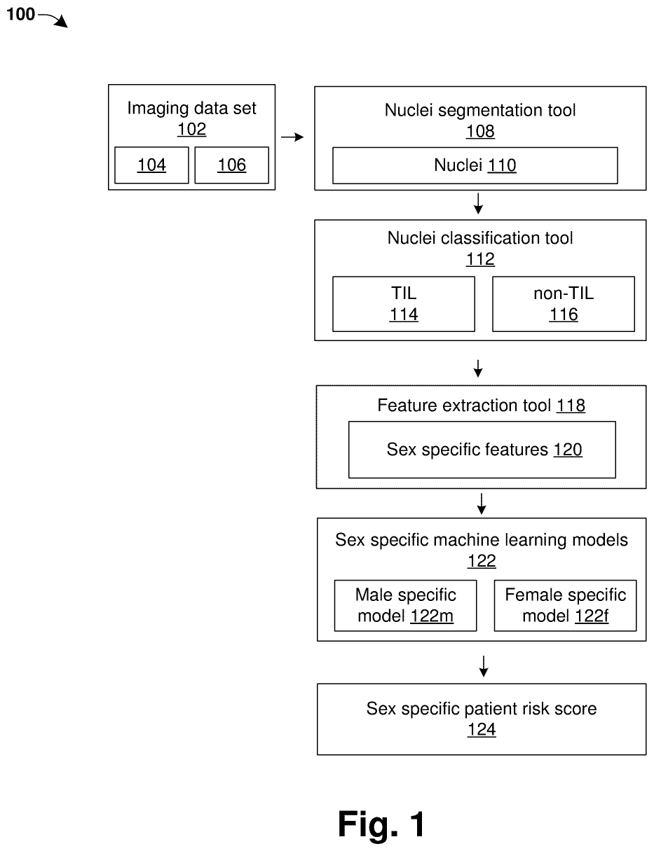

FIG. 1 illustrates some embodiments of a block diagram of a high-grade glioma (HGG) assessment system 100 comprising sex specific machine learning models configured to determine a sex specific patient risk score for a HGG patient.

The HGG assessment system 100 comprises an imaging data set 102 that includes imaging data for a HGG patient (e.g., a patient that has or that has had HGG). In some embodiments, the imaging data set 102 may comprise one or more digitized pathology images (e.g., one or more digitized images of tissue that has been removed from the HGG patient). The one or more digitized pathology images 104 respectively have biological sex information 106 associated with them. For example, each of the one or more digitized pathology images within the imaging data set 102 is associated with a first biological sex (e.g., male) or a second biological sex (e.g., female).

A nuclei segmentation tool 108 is configured to segment the one or more digitized pathology images 104 to generate one or more segmented images that identify nuclei 110 (e.g., cellular nuclei) within respective ones of the one or more digitized pathology images 104. A nuclei classification tool 112 is subsequently configured to classify respective ones of the nuclei 110 as either a lymphocytes or non-lymphocytes. In some embodiments, the nuclei classification tool 112 is configured to classify respective ones of the nuclei 110 as either a tumor infiltrating lymphocyte (TIL) 114 or a non-tumor infiltrating lymphocyte (non-TIL) 116.

A feature extraction tool 118 is configured to operate upon the segmented image to extract a plurality of sex specific features 120. The plurality of sex specific features 120 may be extracted from the nuclei and an environment surrounding the nuclei. In some embodiments, the plurality of sex specific features 120 are spatial features that characterize a spatial organization of the TIL 114 and interactions between the TIL 114 and/or the non-TIL 116. In some additional embodiments, the plurality of sex specific features 120 include density features related to the TIL 114. In some embodiments, the plurality of sex specific features 120 are selected to be prognostic of overall survival for a patient having a specific biological sex. For example, the feature extraction tool 118 may be configured to access the sex information associated with a digitized image and based on the biological sex information 106 to extract either a first set of sex specific features that are prognostic of overall survival in a patient having a first sex (e.g., male) or a second set of sex specific features that are prognostic of overall survival in a patient having a second sex (e.g., female).

The HGG assessment system 100 further comprises sex specific machine learning models 122 configured to utilize the plurality of sex specific features 120 to generate a sex specific patient risk score 124. In various embodiments, the sex specific machine learning models 122 may comprise a male specific machine learning model 122m and a female specific machine learning model 122f. The male specific machine learning model 122m is configured to generate a sex specific patient risk score 124 for male patients, while the female specific machine learning model 122f is configured to generate a sex specific patient risk score 124 for female patients.

During operation, the plurality of sex specific features 120 are provided one of the sex specific machine learning models 122 based on the biological sex information 106. For example, sex specific features 120 corresponding to a first digitized pathology image that is identified as corresponding to a male by the biological sex information 106 will be provided to the male specific machine learning model 122m. Alternatively, sex specific features 120 corresponding to a second digitized pathology image that is identified as corresponding to a female by the biological sex information 106 will be provided to the female specific machine learning model 122f.

By characterizing a positional organization of TILs, as well as interplay between TILs and non-TILs, the sex specific features 120 can represent immunological microenvironments within HGG tumors of patients having different biological sexes (e.g., male, female). From the immunological representations, the sex specific machine learning models 122 are able to represent immunological variances between patients having different biological sexes, thereby enabling the sex specific machine learning models 122 to generate a sex specific patient risk score 124 that accurately provides a prognosis of overall survival in a HGG patient.

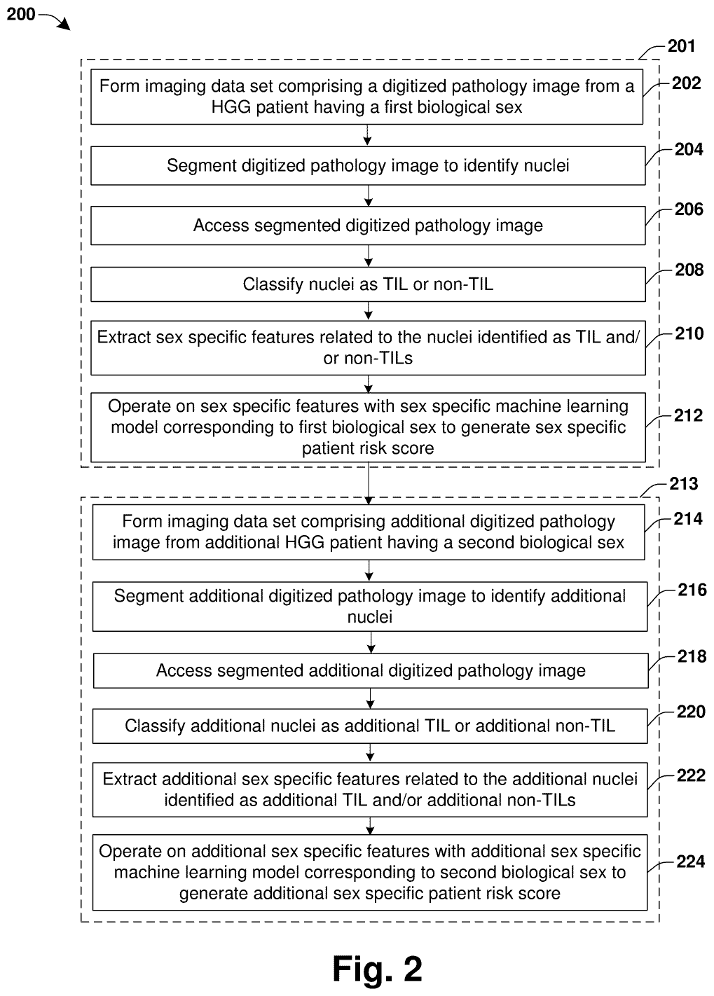

FIG. 2 illustrates some embodiments of a flow diagram showing a method 200 for using a sex specific machine learning model to determine a sex specific patient risk score for a HGG patient.

While the disclosed methods (e.g., methods 200 and 1000) are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 202, an imaging data set is formed to comprise a digitized pathology image from a HGG patient having a first biological sex. In some embodiments, the digitized pathology image may comprise a digitized H&E slide of surgically resected HGG tissue.

At act 204, the digitized pathology image is segmented to identify nuclei within the digitized pathology image.

At act 206, the segmented digitized pathology image is accessed.

At act 208, the nuclei are classified as TILs and non-TILs.

At act 210, sex specific features related to the nuclei identified as TIL and/or non-TIL are extracted. In some embodiments, the sex specific features may relate to structural and/or organizational features of the TILs and/or the non-TILs. For example, the sex specific features may comprise a positional organization of the TILs within a tumor microenvironment and/or a connectivity of nuclei centroids and corresponding cell cluster graphs that are extracted from the TILs, the non-TILs, and interactions therebetween.

At act 212, the sex specific features are operated upon by a sex specific machine learning model (e.g., a sex specific regression model) corresponding to the first biological sex to generate a sex specific patient risk score for the HGG patient.

It will be appreciated that the disclosed method 200 may be applied to different patients having different biological sexes at different times. For example, in some embodiments, the method may be operated in a first instance 201 on a HGG patient having a first biological sex (e.g., male) and in a second instance 213 on an additional HGG patient having a second biological sex (e.g., female). In some embodiments, the first instance 201 may be performed according to acts 202-212, while the second instance 213 may be performed according to acts 214-224.

At act 214, an imaging data set is formed to comprise an additional digitized pathology image from an additional HGG patient having a second biological sex.

At act 216, the additional digitized pathology image is segmented to identify additional nuclei within the additional digitized pathology image.

At act 218, the segmented additional digitized pathology image is accessed.

At act 220, the additional nuclei are classified as additional TILs and additional non-TILs.

At act 222, additional sex specific features related to the nuclei identified as additional TIL and additional non-TIL are extracted.

At act 224, the additional sex specific features are operated upon by an additional sex specific machine learning model corresponding to the second biological sex to generate an additional sex specific patient risk score for the additional HGG patient.

Therefore, the disclosed method utilizes the sex specific features as an immune based biomarker that accounts for epidemiological evidence of sexual dimorphism in HGG. The biomarker is effective, as males in preclinical models have shown more positive responses than females to treatments in development that target the immune system. Based on clinical and biologic observations, the disclosed sex specific approach to the characterization of immune organization within a tumor is able to account for differences in the underlying immune biology, thereby allowing for improved prognosis (e.g., survival prediction and/or stratification of patient risk) for a patient. The improved prognosis may give health care professionals improved guidance on patient treatment. For example, the more accurate prognosis may give health care professionals guidance on recommending that eligible HGG patients enroll in clinical trials and/or the pursuit of chemotherapy alternatives, such as the more-promising immunotherapy-based treatment approaches.

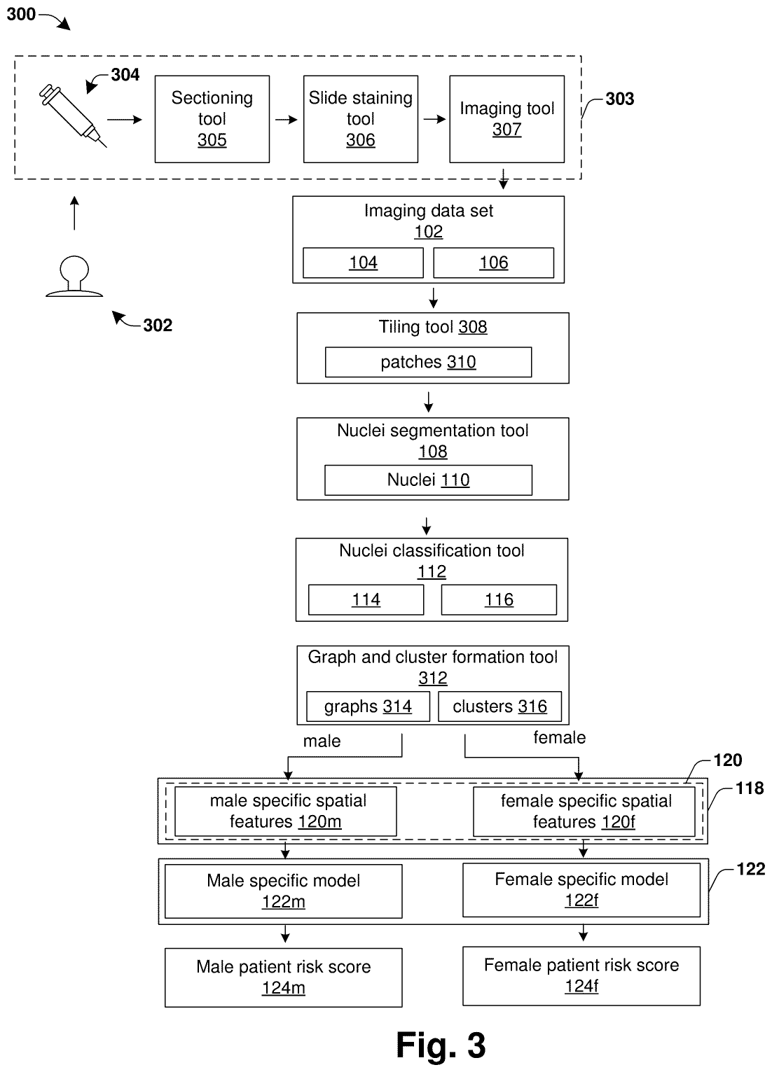

FIG. 3 illustrates some additional embodiments of a block diagram of a HGG assessment system 300 comprising sex specific machine learning models configured to determine a sex specific patient risk score for a HGG patient.

The HGG assessment system 300 comprises an imaging data set 102 having one or more digitized pathology images 104 from one or more HGG patients 302. In some embodiments, the one or more digitized pathology images 104 may comprise digitized images of stained biopsy slides. For example, the one or more digitized pathology images 104 may comprise whole slide images (WSIs) of digitized H&E (Hematoxylin and Eosin) stained slides. The one or more digitized pathology images 104 within the imaging data set 102 respectively have biological sex information 106 associated with them.

In some embodiments, the one or more digitized pathology images 104 may be generated by an image generation stage 303. The image generation stage 303 may be configured to form the one or more digitized pathology images 104 by performing a surgical resection 304 on one of the one or more HGG patients 302 to obtain a tissue sample comprising HGG tissue. A sectioning tool 305 is configured to slice the tissue sample into thin slices that are placed on transparent slides (e.g., glass slides). A slide staining tool 306 is configured to stain the slices on the transparent slides to generate pathology slides. The pathology slides are subsequently converted to the digitized pathology images 104 (e.g., whole slide images (WSI)) by an imaging tool 307 (e.g., comprising a CMOS image sensor, a CCD camera, or the like). In some embodiments, only digitized pathology images 104 corresponding to unique tissue slices are considered for downstream modeling (e.g., by male specific machine learning model 122m or female specific machine learning model).

In some embodiments, a tiling tool 308 is configured to separate the one or more digitized pathology images 104 into a plurality of non-overlapping patches 310. The plurality of non-overlapping patches 310 may extend over an entirety of the one or more digitized pathology images 104. Respective ones of the plurality of non-overlapping patches 310 may comprise HGG tissue and/or non-HGG tissue. In some embodiments, respective ones of the plurality of non-overlapping patches 310 that do not contain at least 50% of HGG tissue without imaging artifacts (e.g., tissue of a resected HGG tumor) may be excluded from downstream operations (e.g., segmentation, classification, etc.). In some embodiments, the plurality of non-overlapping patches 310 may respectively have a size of approximately 250 pixels×250 pixels, 512 pixels×512 pixels, 2048 pixels×2048 pixels, or other similar values. In some embodiments, the tiling tool 308 may further perform color normalization on the plurality of non-overlapping patches 310. In some embodiments, the tiling tool 308 may be implemented as computer code run by a processing unit (e.g., a central processing unit including one or more transistor devices configured to operate computer code to achieve a result, a microcontroller, or the like).

A nuclei segmentation tool 108 is configured to segment the plurality of non-overlapping patches 310, so as to identify nuclei 110. In some embodiments, the nuclei segmentation tool 108 may comprise a first machine learning model (e.g., a deep learning model), which is configured to segment the plurality of non-overlapping patches 310 to identify the nuclei 110.

A nuclei classification tool 112 is configured to subsequently classify the nuclei 110 as a tumor infiltrating lymphocyte (TIL) 114 or a non-tumor infiltrating lymphocyte (non-TIL) 116. In some embodiments, the nuclei classification tool 112 may comprise a support vector machine that utilizes a watershed approach to classify the nuclei 110 as TIL or non-TIL. In other embodiments, the nuclei classification tool 112 may comprise an extreme learning machine or the like.

A graph and cluster formation tool 312 is configured to form graphs 314 (e.g., cell graphs) and nuclei clusters 316 for TIL 114 and for non-TIL 116. The graphs 314 and/or nuclei clusters 316 may characterize a spatial arrangement of the TIL 114 and/or the non-TIL 116. In some embodiments, the graph and cluster formation tool 312 may separately construct graphs for TILs and non-TIL using a decaying function of Euclidean distance to define connections between nuclei centroids. In some embodiments, edges may be added to a graph using one or more thresholds respectively having one or more threshold values. Applying a threshold may produce a set of graphs that form clusters as the network components of each cell type. In some embodiments, the graph and cluster formation tool 312 may be implemented as computer code run by a processing unit (e.g., a central processing unit including one or more transistor devices configured to operate computer code to achieve a result, a microcontroller, or the like).

A feature extraction tool 118 is configured to extract a plurality of sex specific features 120 from the graphs 314 and nuclei clusters 316. In some embodiments, the plurality of sex specific features 120 may comprise spatial features (e.g., graph-based features) extracted from the graphs 314 and the nuclei clusters 316. In some additional embodiments, the plurality of sex specific features 120 may further comprise TIL density features and other TIL metrics relating to area ratio, grouping factor, density matrix value, etc. In various embodiments, the plurality of sex specific features 120 may comprise a plurality of male specific spatial features 120m or a plurality of female specific spatial features 120f. In some embodiments, the feature extraction tool 118 may be implemented as computer code run by a processing unit (e.g., a central processing unit including one or more transistor devices configured to operate computer code to achieve a result, a microcontroller, or the like).

In some embodiments, the plurality of male specific spatial features 120m are features that are selected as being highly (e.g., most) prognostic of overall survival in male patients. In some embodiments, the plurality of male specific spatial features 120m may comprise features related to one or more of an area of opposing cluster intersection, a graph disorder of non-TILs, a value of a TIL density matrix, a non-TIL cluster area, a non-TIL graph triangle area, and a percent of non-TIL clusters surrounding TIL clusters.

The plurality of male specific spatial features 120m are provided to a male specific machine learning model 122m. The male specific machine learning model 122m is a machine learning model (e.g., a regression model, a Cox Hazard regression model, a support vector machine, a linear discriminant analysis (LDA) classifier, a naïve Bayes classifier, or the like) that is being and/or that has been trained on the plurality of male specific spatial features 120m. The male specific machine learning model 122m is configured to calculate a male patient risk score 124m that is indicative of overall survival of a male HGG patient. In some embodiments, the male patient risk score 124m may be calculated using a linear combination of top feature coefficients and feature values.

In some embodiments, the plurality of female specific spatial features 120f are features that are selected as being highly (e.g., most) prognostic of overall survival in female patients. In some embodiments, the plurality of female specific spatial features 120f may comprise features related to one or more of a ratio of an area of opposing cluster intersection to the area of TILs and the percent of non-TIL clusters surrounding other non-TIL clusters.

The plurality of female specific spatial features 120f are provided to a female specific machine learning model 122f. The female specific machine learning model 122f is a regression model that is being and/or that has been trained on the plurality of female specific spatial features 120f. The female specific machine learning model 122f is configured to calculate a female patient risk score 124f that is indicative of overall survival of a female patient.

In some embodiments, the disclosed sex specific patient risk score (e.g., male patient risk score 124m and/or the female patient risk score 124f) may comprise a number denoting a risk of death (e.g., a numerical value that is indicative of an overall survival associated with a HGG patient). In some embodiments, lower sex specific patient risk score scores may be assigned to males generally having higher positional mixing of TILs and non-TILs clusters and higher variability in density of TILs and non-TILs, while lower sex specific patient risk score may be assigned to females generally having higher coverage of TIL clusters that did not intersect non-TIL clusters and higher proportions of non-TIL clusters surrounding other non-TIL clusters.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

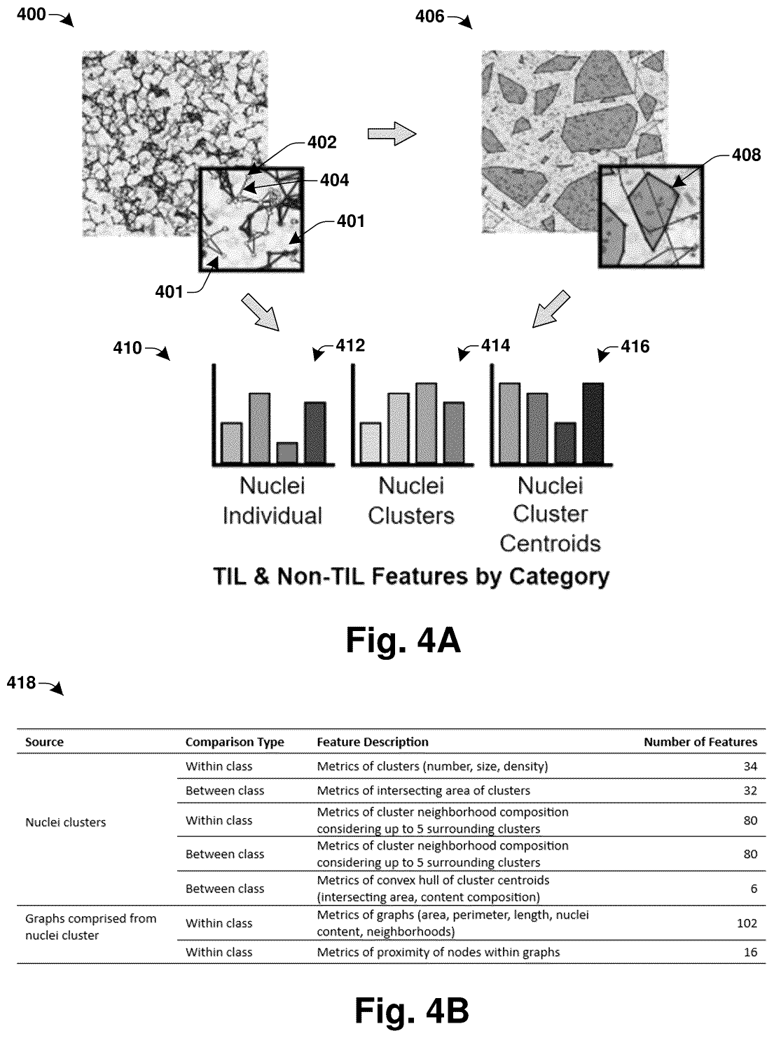

FIG. 4A some embodiments of a workflow for generating sex specific features from a digitized pathology image.

Image 400 illustrates an example tile with graphs 401 illustrated for nuclei classified as TILS and non-TILs. The graphs 401 are constructed separately for TILs and non-TILs using nuclei centroids 402 of a respective cell type as nodes. Edges 404 are disposed between the nuclei centroids 402. Using the nuclei centroids 402 and edges 404, a spatial arrangement between the nuclei centroids 402 (e.g., TIL and/or non-TIL) may be characterized by a plurality of features.

In some embodiments, a decaying function of Euclidean distance may be employed to limit the edges 404 connecting nuclei centroids 402 to cells located proximally to each other. In some such embodiments, edges 404 are added to a graph 401 based on a function's threshold (e.g., of 94 pixels for slides imaged at 40× magnification and 47 pixels for slides imaged at 20× magnification, corresponding to approximately 24 μm). Applying a threshold produces separate sets of TIL and non-TIL graphs, from which network components are taken to define nuclei clusters.

Image 406 illustrates an example tile with nuclei clusters 408 illustrated for nuclei classified as TILs and non-TILs. In some embodiments, the nuclei clusters 408 may comprise polygonal clusters formed to comprise proximally situated nuclei identified in the graphs 401.

A plurality of sex specific features 410 are extracted from the graphs 401 and the nuclei clusters 408. The plurality of sex specific features 410 comprise mathematical descriptors of both the graphs 401 and the nuclei clusters 408. The plurality of sex specific features 410 may comprise one or more sex specific features (e.g., TIL density and other TIL metrics relating to area ratio, grouping factor, density matric value, etc.) relating to individual nuclei 412, one or more sex specific spatial features relating to nuclei clusters 414 (e.g., TIL and non-TIL nuclei clusters), and/or one or more sex specific spatial features relating to graphs that use nuclei cluster centroids 416 (e.g., TIL and non-TIL graphs based on nuclei cluster centroids).

For example, in some embodiments, using the graphs 401 and nuclei clusters 408, three hundred and fifty (350) sex specific spatial features may be calculated to represent spatial arrangement and co-localization of TILs and non-TILs. Additionally, nineteen (19) sex specific density features related to TIL density features may be extracted. To represent a given patient, summary statistics may be calculated for each of the sex specific spatial and density features across all processed tiles. The metrics included total, mean, standard deviation, median, maximum, minimum, skewness, and kurtosis resulting in 2,952 sex specific spatial and density features per patient. From the 2,952 sex specific spatial and density features, a set of sex specific features (e.g., less than 2,952 sex specific features) may be selected to be provided to a downstream sex specific machine learning model.

The basis of the analyzed constructs used to generate the sex specific spatial features is either the graphs 401 or the nuclei clusters 408. Considering TIL and non-TIL classes, measurements may be both within and between cell type classes. For example, FIG. 4B illustrates a graph 418 showing sex specific spatial features including features extracted from nuclei clusters, features extracted from graphs, and features extracted between the nuclei clusters and graphs.

In some embodiments, the sex specific density features may comprise and/or include one or more of a number of TILs/Tissue Area, TIL Total Area/Tissue Area, a number of TILs/Total Nuclei, Max TIL Grouping Factor, Min TIL Grouping Factor, Avg TIL Grouping Factor, Std TIL Grouping Factor, Median TIL Grouping Factor, Mode TIL Grouping Factor, Num Highly Grouped TIL, a number of TIL/Total Cony Hull Area, a TIL Cony Hull Area/Total Cony Hull Area, Intersected Area Cony Hull TIL & Non-TIL, Max Density Matrix Val, Min Density Matrix Val, Avg Density Matrix Val, Std Density Matrix Val, Median Density Matrix Val, and Mode Density Matrix Val. In some embodiments, the sex specific density features may also include TIL count.

In some embodiments, the features selected for male HGG patients may be related to individual TIL nuclei (e.g., TIL density features), TIL and non-TIL nuclei clusters, and non-TIL graphs based on nuclei cluster centroids. For individual nuclei, higher measures of TIL density matrix value may be favorable. Beneficial features related to nuclei clusters may include higher intersection of TIL and non-TIL cluster area, lower non-TIL cluster area, and higher percent of non-TIL clusters surrounding TILs within 5 nearest neighbors. Lastly, non-TIL graphs based on nuclei cluster centroids that demonstrate higher disorder considering 7 nearest neighbors and higher graph triangle area may be identified as positive attributes. Together these features may suggest higher presence of TILs, lower presence of non-TILs, higher spatial interaction among TILs and non-TILs, and increased disorder among non-TILs are beneficial.

In some embodiments, the features selected for female HGG patients may be related to TIL and non-TIL nuclei clusters, but not individual TIL nuclei or non-TIL graphs based on nuclei cluster centroids. A higher ratio of opposing group intersection area to TIL area and a higher percentage of non-TILs surrounding non-TILs within 3 nearest neighbors may be associated with favorable prognosis. These features may suggest benefit to the interaction of TIL clusters with non-TIL clusters within the HGG microenvironment and the disruption of non-TIL organization into several clusters.

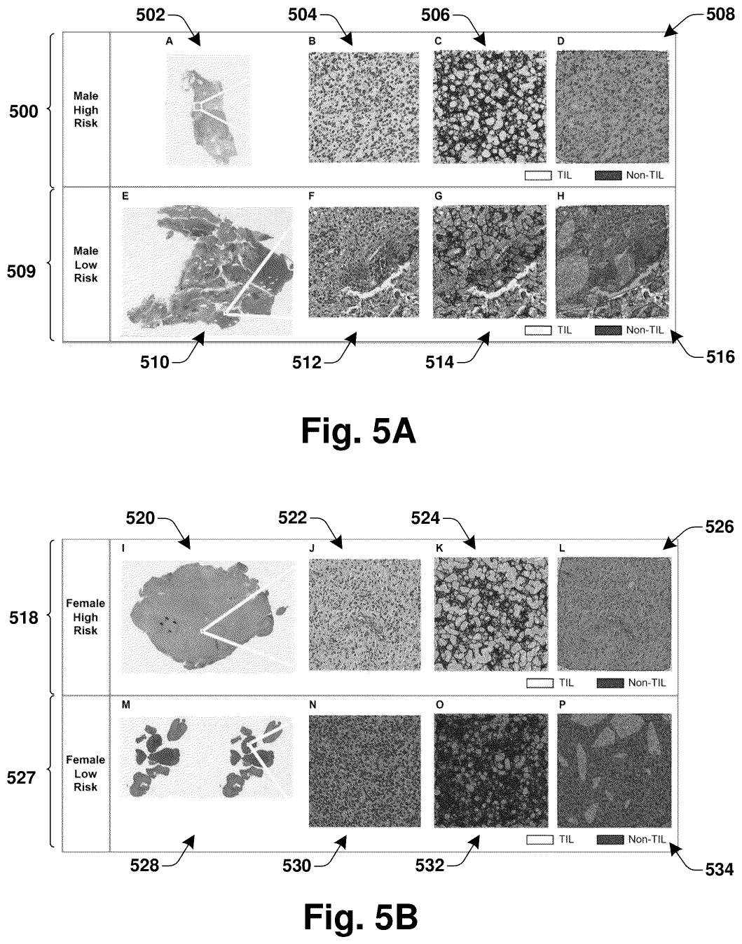

FIGS. 5A-5B illustrate some embodiments of digitized pathology images showing visualizations of prognostic features selected for male and female imaging data sets according to a disclosed method and/or apparatus. As can be seen in FIGS. 5A-5B, an overall survival of a HGG patient may be at least in-part related to a density of TIL within the patient (e.g., overall survival of a HGG patient may be inversely proportional to a density of TIL).

FIG. 5A illustrates digitized pathology images corresponding to a male imaging data set. Images 500 correspond to a male that is classified as high risk (e.g., having a relatively low chance of overall survival). A whole slide image (WSI) 502 corresponding to the male is shown. A tile 504 from the WSI 502 illustrates TIL nuclei outlined in yellow and non-TIL nuclei outlined in blue. Tile 506 shows graphs of TILs in yellow and graphs of non-TILs in blue. Tile 508 shows clusters of TILs in yellow and clusters of non-TILs in blue.

Images 509 correspond to a male that is classified as low risk (e.g., having a relatively high chance of overall survival). A WSI 510 corresponding to the male is shown. A tile 512 from the WSI 510 illustrates TIL nuclei outlined in yellow and non-TIL nuclei outlined in blue. Tile 514 shows graphs of TILs in yellow and graphs of non-TILs in blue. Tile 516 shows clusters of TILs in yellow and clusters of non-TILs in blue.

FIG. 5B illustrates digitized pathology images corresponding to a female imaging data set. Images 518 correspond to a female that is classified as high risk (e.g., having a relatively low chance of overall survival). A WSI 520 corresponding to the female is shown. A tile 522 from the WSI 520 illustrates TIL nuclei outlined in yellow and non-TIL nuclei outlined in blue. Tile 524 shows graphs of TILs in yellow and graphs of non-TILs in blue. Tile 526 shows clusters of TILs in yellow and clusters of non-TILs in blue.

Images 527 correspond to a female that is classified as low risk (e.g., having a relatively high chance of overall survival). A WSI 528 corresponding to the female is shown. A tile 530 from the WSI 528 illustrates TIL nuclei outlined in yellow and non-TIL nuclei outlined in blue. Tile 532 shows graphs of TILs in yellow and graphs of non-TILs in blue. Tile 534 shows clusters of TILs in yellow and clusters of non-TILs in blue.

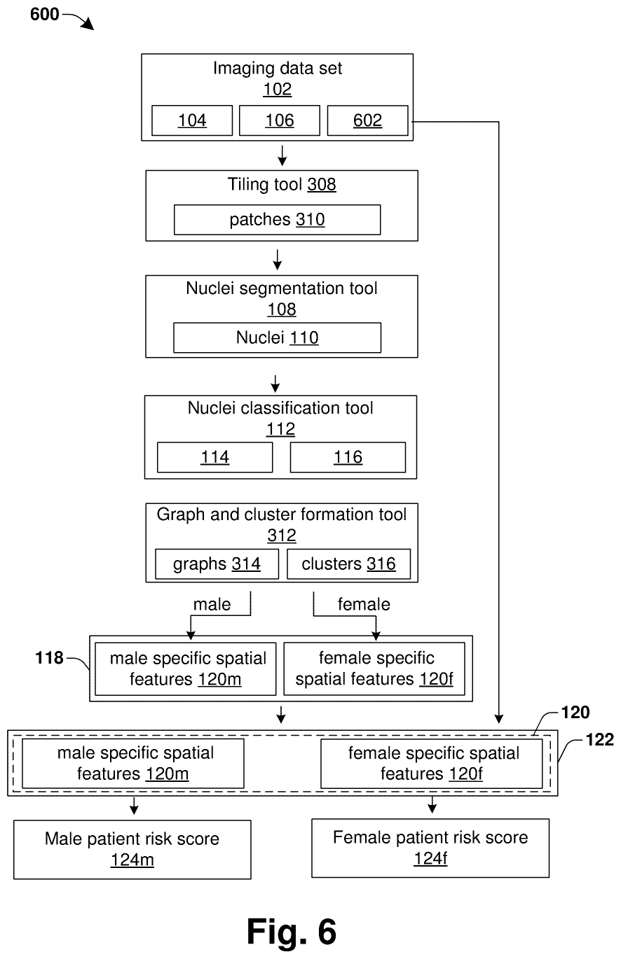

FIG. 6 illustrates a block diagram of some additional embodiments of a HGG assessment system 600 comprising sex specific machine learning models that are configured to determine a prognosis for a patient using sex specific features.

The HGG assessment system 600 comprises an imaging data set 102 including imaging data from one or more HGG patients. The imaging data set 102 includes one or more digitized pathology images 104. The one or more digitized pathology images 104 respectively have both biological sex information 106 and one or more additional prognostic attributes 602 associated with them. In some embodiments, the one or more additional prognostic attributes 602 may comprise an age of a HGG patient and/or an IDH-mutation status of a HGG patient. In some additional embodiments, the one or more additional prognostic attributes 602 may comprise a Karnofsky performance scale (KPS) score of a patient, whether a HGG tumor is unmethylated or methylated, an EGFR wild type or mutated, and/or the like.

A tiling tool 308 is configured to separate the one or more digitized pathology images 104 into a plurality of non-overlapping patches 310. A nuclei segmentation tool 108 is configured to segment one or more of the plurality of non-overlapping patches 310 to identify nuclei 110. A nuclei classification tool 112 is configured to subsequently identify the nuclei 110 as a TIL 114 or a non-TIL 116. A graph and cluster formation tool 312 is configured to form graphs 314 and nuclei clusters 316 for the TIL 114 and the non-TIL 116. A feature extraction tool 118 is configured to extract a plurality of sex specific features 120 from the graphs 314 and nuclei clusters 316.

A sex specific machine learning model (e.g., male specific machine learning model 122m or female specific machine learning model 122f) is configured to operate upon the plurality of sex specific features 120 to generate a sex specific prognosis for the HGG patient. In some additional embodiments, the sex specific machine learning model is further configured to operate upon the one or more additional prognostic attributes 602 to generate the sex specific prognosis for the HGG patient. For example, in some embodiments the sex specific machine learning model may be configured to operate upon both the plurality of sex specific features 120 and a HGG patient's age to determine a sex specific prognosis for the HGG patient. In other embodiments, the sex specific machine learning model may be configured to operate upon both the plurality of sex specific features 120 and a HGG patient's IDH-mutation status to determine a sex specific prognosis for the HGG patient.

It has been appreciated that sex specific machine learning models that operate upon both the plurality of sex specific features 120 (e.g., TIL based spatial features) and the one or more additional prognostic attributes 602 (e.g., age, IDH-mutation status, etc.) are able to provide for improved splits between high and low-risk patient stratification in comparison to sex specific machine learning models that utilize each factor individually.

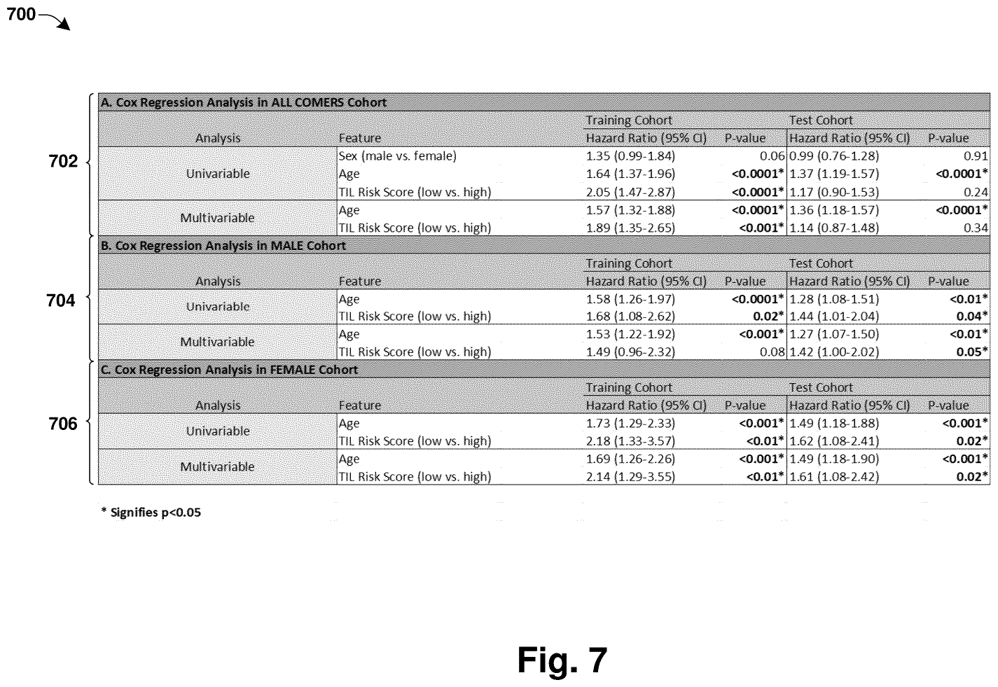

FIG. 7 illustrates a table 700 showing exemplary results of a Cox Regression model for different sex-based models according to a disclosed method and/or apparatus.

The table 700 shows univariable and multivariable survival analysis showing hazard ratios for different sex groups 702-706. For example, the table 700 shows univariable and multivariable survival analysis for a two-sex group 702 (e.g., a group containing male and female members), univariable and multivariable survival analysis for a male sex group 704, and univariable and multivariable survival analysis for a female sex group 706.

The ability of the disclosed machine learning models to generate a sex specific patient risk score that maintains significance for male and female sexes when controlling for age demonstrates an independent value of using sex specific features. Furthermore, from multivariable analysis it can be seen that TIL based features are prognostic independent of age for both the male sex group 704 and the female sex group 706, but not for the two-sex group 702. Rather, the two-sex group 702 has a hazard ratio that generally overestimates survival of male patients and that generally underestimates overall survival of female patients. Therefore, the sex specific machine learning models are able to benefit from the use of the disclosed sex specific features in a way that a sex neutral model cannot.

In some embodiments, the disclosed HGG assessment system may comprise a machine learning model that is configured to take into consideration additional prognostic factors. For example, in table 700 it can be seen that age is a prognostic factor for overall survival in all groups. Since age is a prognostic factor for overall survival, the disclosed sex specific machine learning models (e.g., male specific machine learning model 122m and/or the female specific machine learning model 122f) may be configured to take age into consideration in some embodiments.

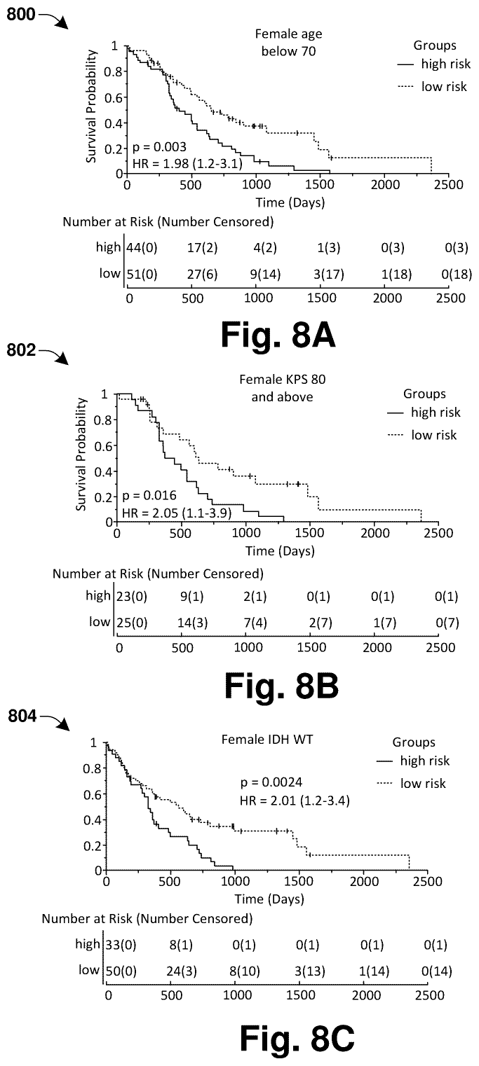

FIGS. 8A-8C illustrate some embodiments of Kaplan-Meier survival curves determined based upon the disclosed method and corresponding to subgroups within a female imaging data set.

FIG. 8A illustrates an exemplary Kaplan Meier survival curve 800 for a subgroup of females having an age of below 70. FIG. 8B illustrates an exemplary Kaplan Meier survival curve 802 for a subgroup of females having a Karnofsky performance scale (KPS) score of 80 or above. FIG. 8C illustrates an exemplary Kaplan Meier survival curve 804 for a subgroup of females without IDH mutations.

From FIGS. 8A-8C it can be seen that IDH-mutation status has a positive prognostic value for overall survival in female patients. Since IDH-mutation status is a prognostic factor for overall survival, the disclosed sex specific machine learning models (e.g., male specific machine learning model 122m and/or the female specific machine learning model 122f) may be configured to take IDH-mutation status into consideration for female patients in some embodiments.

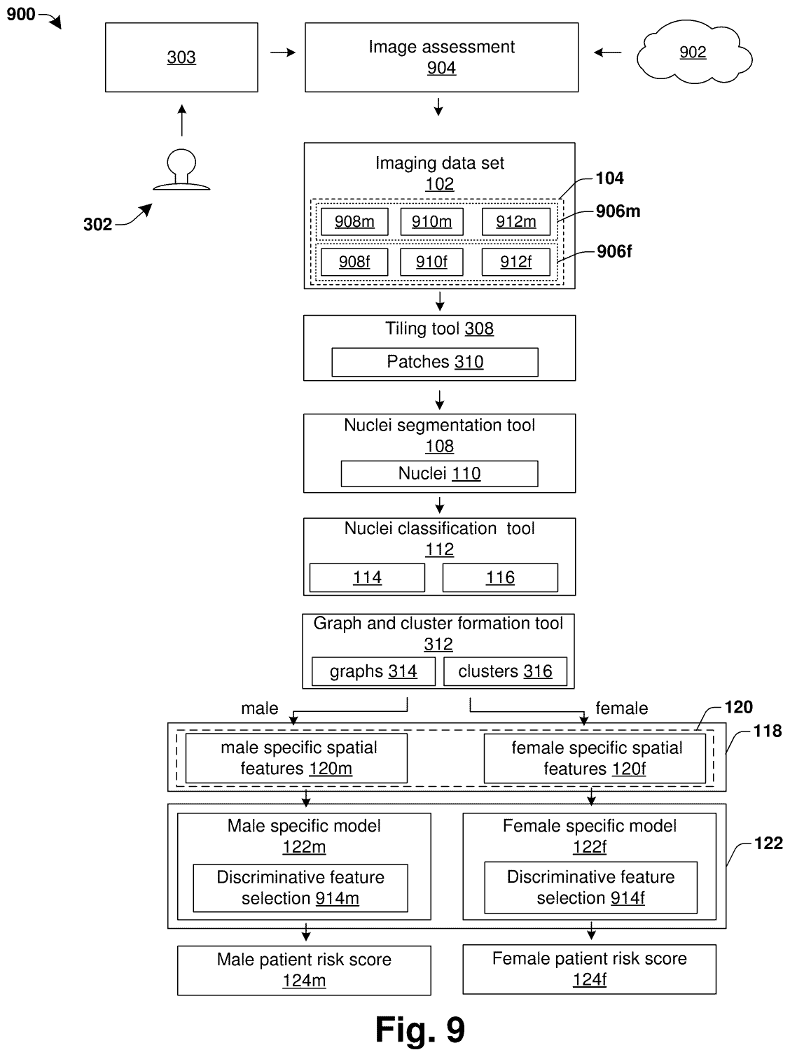

FIG. 9 illustrates a block diagram of some additional embodiments of a HGG assessment system 900 comprising sex specific machine learning models that are configured to be trained to determine a prognosis for a patient using sex specific features extracted from digitized pathology images.

The HGG assessment system 900 comprises an imaging data set 102 including imaging data from a plurality of HGG patients. The imaging data set 102 includes a plurality of digitized pathology images 104. In some embodiments, the plurality of digitized pathology images 104 comprise WSIs of HGG tissue. In various embodiments, the plurality of digitized pathology images 104 may be obtained by an image generation stage 303 and/or from an on-line database 902 and/or archive containing digitized pathology images from patients generated at different sites (e.g., different hospitals, research laboratories, and/or the like). Prior to including digitized pathology images within the imaging data set 102, the digitized pathology images may be subjected to image assessment 904 including one or more inclusion criteria and exclusion criteria.

The imaging data set 102 may include a male training data set 906m and a female training data set 906f. The male training data set 906m comprises digitized pathology images of biological male patients and not of biological female patients. The female training data set 906f comprises digitized pathology images of biological female patients and not of biological male patients. In some embodiments, the male training data set 906m may comprise training data 908m, testing data 910m, and/or validation data 912m. In some embodiments, the female training data set 906f may comprise training data 908f, testing data 910f, and/or validation data 912f.

The plurality of digitized pathology images 104 may be separated into a plurality of non-overlapping patches 310 by a tiling tool 308. A nuclei segmentation tool 108 is configured to segment one or more of the plurality of non-overlapping patches 310 to identify nuclei 110. A nuclei classification tool 112 is configured to subsequently identify the nuclei 110 as TIL 114 or non-TIL 116. A graph and cluster formation tool 312 is configured to form graphs 314 and nuclei clusters 316 for the TIL 114 and the non-TIL 116. A feature extraction tool 118 is configured to extract a plurality of sex specific features 120 from the graphs 314 and nuclei clusters 316. In various embodiments, the plurality of sex specific features 120 may comprise a plurality of male specific spatial features 120m and a plurality of female specific spatial features 120f.

A male specific machine learning model 122m is configured to act upon the plurality of male specific spatial features 120m to generate a male patient risk score 124m. In some embodiments, the male specific machine learning model 122m may comprise a discriminative feature selection component 914m that is configured to identify a plurality of male specific spatial features 120m that are highly (e.g., most) prognostic of overall survival in male patients. In some embodiments, the discriminative feature selection component 914m may give higher prognostic value to spatial features over density features. In some embodiments, the discriminative feature selection component 914m may comprise a least absolute shrinkage and selection operator (LASSO) algorithm. In some embodiments, the extraction of the plurality of male specific spatial features 120m and the calculation of the male patient risk score 124m may be coincidentally performed using a Cox Hazard regression model with least absolute shrinkage and selection operator (LASSO) feature select.

In some embodiments, the male training data set 906m may be used to train initial versions of the male specific machine learning model. The initial versions of the male specific machine learning model may be subsequently fine-tuned using the testing data 910m to generate one or more evaluation models. The validation data 912m may then be used to generate the male specific machine learning model 122m from the one or more evaluation models. A prognostic value of the male patient risk score 124m may be subsequently assessed by Kaplan Meier estimates.

A female specific machine learning model 122f is configured to act upon the plurality of female specific spatial features 120f to generate a female patient risk score 124f. In some embodiments, the female specific machine learning model 122f may comprise a discriminative feature selection component 914f that is configured to identify a plurality of female specific spatial features 120f that are highly (e.g., most) prognostic of overall survival in female patients. In some embodiments, the discriminative feature selection component 914f may give higher prognostic value to spatial features over density features. In some embodiments, the discriminative feature selection component 914f may comprise a LASSO algorithm. In some embodiments, the extraction of the plurality of female specific spatial features 120f and the calculation of the female patient risk score 124f may be coincidentally performed using a Cox Hazard regression model with a LASSO feature select.

The female training data set 906f may be used to train initial versions of a female specific machine learning model. The initial versions of the female specific machine learning model may be subsequently fine-tuned using the testing data 910f to generate one or more evaluation models. The validation data 912f may then be used to generate the female specific machine learning model 122f from the one or more evaluation models. A prognostic value of the female patient risk score 124f may be subsequently assessed by Kaplan Meier estimates.

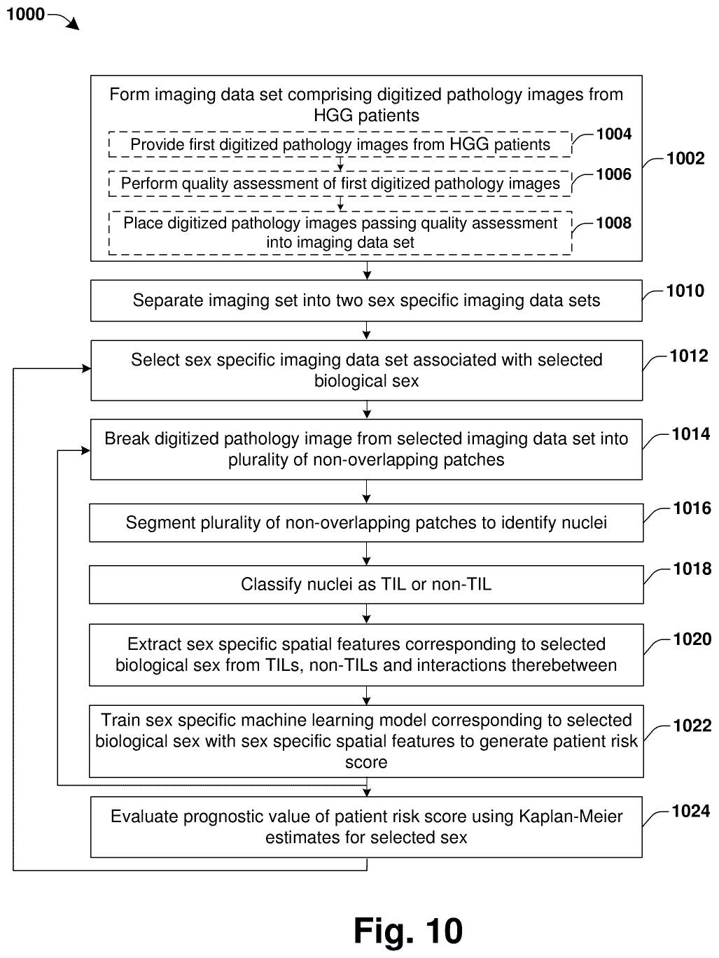

FIG. 10 illustrates some embodiments of a flow diagram showing a method 1000 for training a sex specific machine learning model to determine a sex specific patient risk score using sex specific features extracted from digitized pathology images.

At act 1002, an imaging data set is formed to include one or more digitized pathology images from HGG patients. In some embodiments, the imaging data set may be formed according to acts 1004-1008.

At act 1004, first digitized pathology images from HGG patients are provided. The first digitized images may comprise digitized H&E slides from surgically resected HGG tissue.

At act 1006, a quality assessment of the first digitized pathology images is performed.

At act 1008, digitized pathology images passing the quality assessment are used to form the imaging data set.

At act 1010, the imaging data set may be separated into two sex specific imaging data sets (e.g., into a male imaging data set and/or a female imaging data set).

At act 1012, a sex specific imaging data set associated with a selected biological sex is selected.

At act 1014, a digitized pathology image from the selected sex specific imaging data set is broken into a plurality of non-overlapping patches.

At act 1016, the plurality of non-overlapping patches are segmented to identify nuclei.

At act 1018, the nuclei are classified as TIL and non-TIL.

At act 1020, sex specific features corresponding to the selected biological sex are extracted from the TILs, the non-TILs, and/or interactions therebetween.

At act 1022, a sex specific machine learning model corresponding to the selected biological sex is trained using the sex specific features to generate a sex specific patient risk score.

In some embodiments, acts 1014-1022 may be iteratively performed on different digitized pathology images from the selected imaging data set to train the sex specific machine learning model corresponding to the selected biological sex.

At act 1024, a prognostic value of the patient risk score is evaluated using Kaplan-Meier estimates for an associated biological sex.

In some additional embodiments after a first sex specific machine learning model is trained, acts 1012-1024 may be performed on different digitized pathology images from a different sex specific imaging data set to train a different sex specific machine learning model corresponding to a different selected biological sex. For example, acts 1012-1024 may be performed a first time to train a female specific machine learning model and then performed a second time to train a male specific machine learning model.

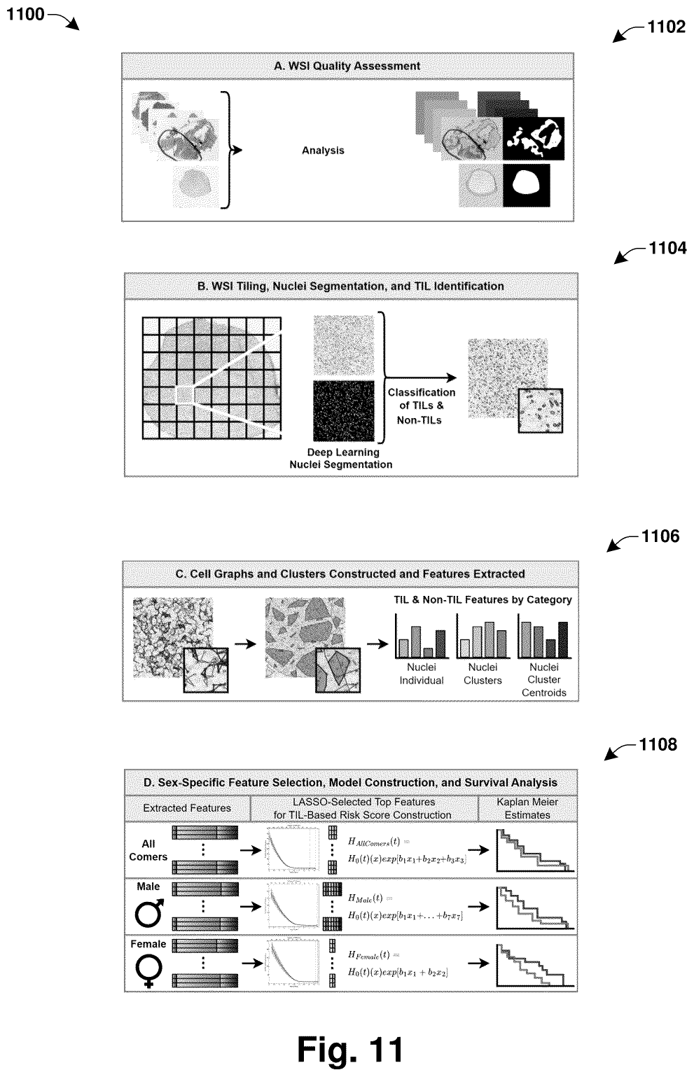

FIG. 11 illustrates some embodiments of a block diagram 1100 showing a workflow of the disclosed method for using a sex specific machine learning model to determine a sex specific patient risk score.

In block 1102, a quality assessment comprising one or more quality assessment operations is performed on a digitized whole slide image (WSI). In some embodiments, the quality assessment operations may utilize a quality control tool for digital pathology slides (e.g., HistoQC) to compute relevant quality control metrics for the plurality of digitized images within an imaging data set.

In block 1104, a whole slide image is broken into a plurality of tiles. The tiles are subsequently segmented to identify individual nuclei. In some embodiments, the segmentation may be performed using a deep learning model. The nuclei are then classified as TIL or non-TIL. In some embodiments, the classification may be performed using a support vector machine.

In block 1106, graphs and nuclei clusters are generated for the nuclei within the plurality of tiles. Spatial features related to a connectivity of the graphs and the nuclei clusters are then extracted for TILs, non-TILs, and interactions therebetween.

In block 1108, sex specific features are identified from the extracted spatial features. The sex specific features may be identified using a LASSO algorithm. A prognostic value of a patient risk score is subsequently evaluated using Kaplan-Meier estimates.

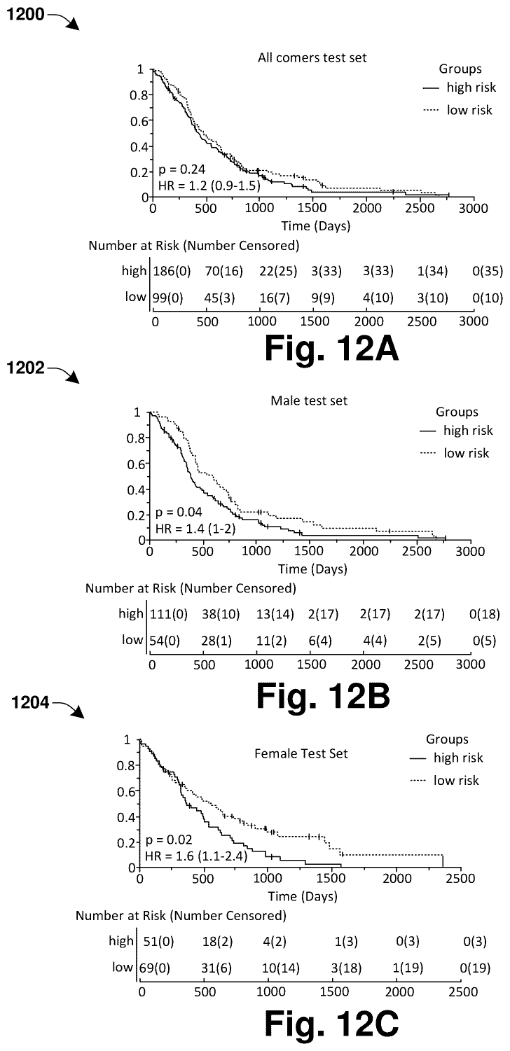

FIGS. 12A-12C illustrate some embodiments of Kaplan Meier survival curves corresponding to different sex-based models for determining overall survival for HGG.

The Kaplan-Meier survival curves 1200-1204 are characterized by the Cox regression models using the LASSO selected TIL-based features. The Kaplan-Meier survival curves of the high-risk groups are shown in red and Kaplan-Meier survival curves of the low-risk are shown in blue. As shown in graph 1200 of FIG. 12A, disclosed models were applied to independent test sets corresponding to a data set comprising male and female imaging data to achieve HR=1.17, 95% CI=0.90-1.57, p=0.24. As shown in graph 1202 of FIG. 12B, disclosed models were applied to independent test sets corresponding to a male imaging data set cohort to achieve HR=1.44, 95% CI=1.01-2.04, p=0.042. As shown in graph 1204 of FIG. 12C, disclosed models were applied to independent test sets corresponding to a female imaging data set to achieve HR=1.615, 95% CI=1.08-2.41, p=0.019. The P values are two-sided and are calculated using log-rank test. Hazard ratios (HR) are provided with corresponding 95% confidence interval. The result of the Kaplan-Meier survival curves 1200-1204 show that the sex specific machine learning models provide for good prognostic value in comparison to gender neural models.

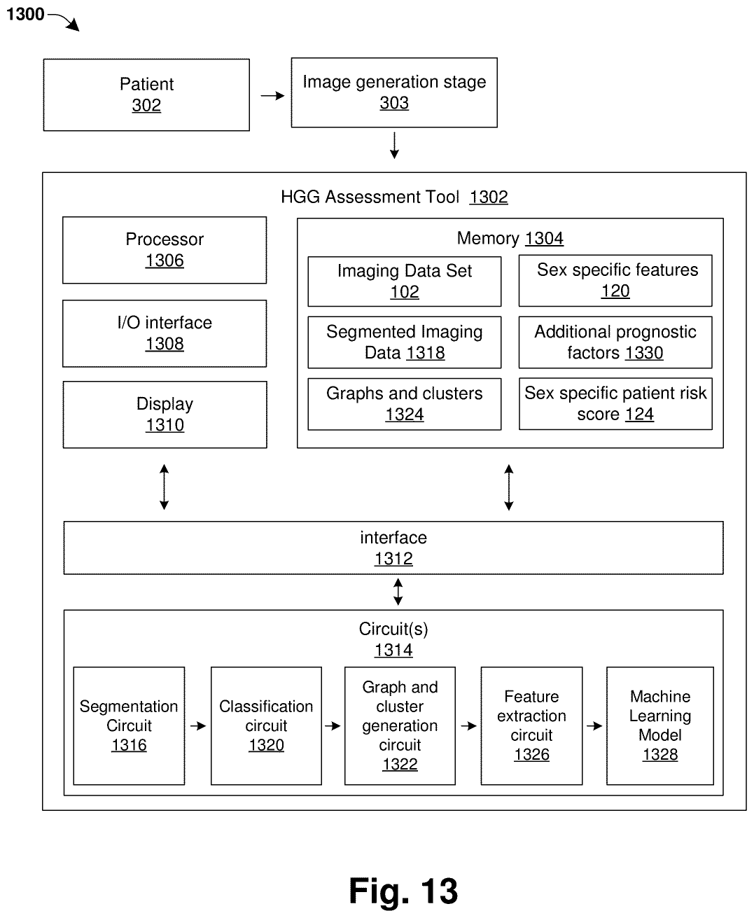

FIG. 13 illustrates a block diagram of some embodiments of a prognostic apparatus 1300 comprising a disclosed HGG assessment tool.

The prognostic apparatus 1300 comprises an HGG assessment tool 1302. The HGG assessment tool 1302 is coupled to an image generation stage 303 that is configured to generate one or more digitized pathology images (e.g., WSIs) of pathology slides corresponding to a HGG patient 302 (e.g., a patient that has or that has had HGG).

The HGG assessment tool 1302 comprises a processor 1306 and a memory 1304. The processor 1306 can, in various embodiments, comprise circuitry such as, but not limited to, one or more single-core or multi-core processors. The processor 1306 can include any combination of general-purpose processors and dedicated processors (e.g., graphics processors, application processors, etc.). The processor 1306 can be coupled with and/or can comprise memory (e.g., memory 1304) or storage and can be configured to execute instructions stored in the memory 1304 or storage to enable various apparatus, applications, or operating systems to perform operations and/or methods discussed herein.

Memory 1304 can be further configured to store an imaging data set 102 comprising the one or more digitized pathology images (e.g., digitized WSIs) containing HGG tissue. The one or more digitized pathology images include a plurality of imaging units (e.g., pixels, voxels, etc.) respectively having an associated intensity pixels. In some additional embodiments, the one or more digitized pathology images may be stored in the memory 1304 as one or more training sets, testing sets, and/or validation sets of digitized pathology images for training a machine learning circuit.

The HGG assessment tool 1302 also comprises an input/output (I/O) interface 1308 (e.g., associated with one or more I/O devices), a display 1310, one or more circuits 1314, and an interface 1312 that connects the processor 1306, the memory 1304, the I/O interface 1308, the display 1310, and the one or more circuits 1314. The I/O interface 1308 can be configured to transfer data between the memory 1304, the processor 1306, the one or more circuits 1314, and external devices (e.g., image generation stage 303).

In some embodiments, the one or more circuits 1314 may comprise hardware components. In other embodiments, the one or more circuits 1314 may comprise software components. In such embodiments, the one or more circuits 1314 may execute code stored in the memory 1304. The one or more circuits 1314 can comprise a segmentation circuit 1316 configured to segment respective ones of the one or more digitized pathology images to generate segmented imaging data 1318 that identifies nuclei within the one or more digitized pathology images. In some embodiments, the segmentation circuit 1316 may comprise a deep learning model/circuit.

In some additional embodiments, the one or more circuits 1314 may further comprise a classification circuit 1320. In some embodiments, the classification circuit 1320 is configured to classify the nuclei as TIL and/or non-TIL. In some embodiments, the classification circuit 1320 may comprise a support vector machine configured to operate a support vector machine algorithm to classify the nuclei.

In some additional embodiments, the one or more circuits 1314 may further comprise a graph and cluster generation circuit 1322. In some embodiments, the graph and cluster generation circuit 1322 is configured to operate upon the TIL and/or non-TIL to generate graphs and clusters 1324.

In some additional embodiments, the one or more circuits 1314 may further comprise a feature extraction circuit 1326. In some embodiments, the feature extraction circuit 1326 is configured to extract a plurality of sex specific features from the graphs and clusters 1324. In some embodiments, the plurality of sex specific features may comprise features describing spatial characteristics of the TIL and the non-TIL. In some additional embodiments, the plurality of sex specific features may comprise TIL density features and other TIL metrics relating to area ratio, grouping factor, density matric value, etc.

In some additional embodiments, the one or more circuits 1314 may further comprise a sex specific machine learning circuit 1328. In some embodiments, the sex specific machine learning circuit 1328 is configured to utilize the plurality of sex specific features 120 to generate a sex specific patient risk score 124. In some additional embodiments, the sex specific machine learning circuit 1328 may also be configured to take into consideration one or more additional prognostic factors 1330 such as age, IDH mutation, and/or the like, in generating the sex specific patient risk score 124.

### Example Use Case:

Methods: A multi-site cohort of N=486 patients consisting of digital images of Hematoxylin and Eosin (H&E) slides of resected HOG tumors was established using publicly available repositories. The training set is comprised of N-201 patients from twelve contributing sites of the Cancer Genome Atlas (TOGA). The validation set is comprised of N=285 patients combining cases from two hold-out TOGA sites, the Clinical Proteomic Tumor Analysis Consortium, and the Ivy Glioblastoma Atlas Project. Each whole slide image of the H&E slides underwent quality assessment, patch extraction, and nuclei segmentation. Individual nuclei were then labeled as TIL and non-TIL cells by employing a pre-trained classifier. Spatial features related to the connectivity of nuclei centroids and corresponding cell cluster graphs were extracted for TILs, non-TILs, and between-group interactions. These TIL-centric histological features were fed into a Cox Hazard regression model with least absolute shrinkage and selection operator feature selection, to stratify patients as low or high-risk. Survival models were evaluated using Kaplan-Meier estimates, across male, female, and combined cohorts.

Results: Selected TIL-based features were significantly associated with OS in the test set for the male (hazard ratio (HR)=1.44, 95% confidence interval (CI)=1.01-2.04, p=0.042) and female (HR=1.615, 95% CI=1.08-2.41, p=0.019) cohorts. The cohort combining male and female patients did not yield significant survival risk stratification. Multivariable survival analysis demonstrated prognostic value of the sex-specific TIL-based features independent of age for both the male (HR=1.42, 95% CI=1.00-2.02, p=0.0498) and female (HR=1.611, 95% CI=1.08-2.42, p=0.021) cohorts.

Conclusion: Our findings suggest that sex-specific TIL organization histological features may be prognostic of OS in HGG tumors.

Therefore, the present disclosure relates to a method and associated apparatus for determining a prognosis for a patient having HGG using sex specific features extracted from digitized pathology images of HGG tissue excised from the patient. The disclosed method and associated apparatus are able to achieve an improved performance over traditional manual assessments because the sex specific features that the disclosed method and apparatus extracts from the digitized pathology images are at a higher order or higher level than a human can resolve in the human mind or with pencil and paper.

In some embodiments, the present disclosure relates to a method. The method includes accessing a segmented digitized pathology image corresponding to a high grade glioma (HGG) patient having a first biological sex, the segmented digitized pathology image identifying nuclei; classifying the nuclei as tumor infiltrating lymphocytes (TILs) or non-TILs; extracting sex specific features related to the nuclei identified as the TILs and the non-TILs, wherein the sex specific features characterizing a spatial organization of the TILs and the non-TILs; and operating on the sex specific features with a sex specific machine learning model corresponding to the first biological sex to generate a sex specific patient risk score.

In other embodiments, the present disclosure relates to a non-transitory computer-readable medium storing computer-executable instructions that, when executed, cause a processor to perform operations, including forming an imaging data set having a digitized pathology image of a patient having or having had high grade glioma (HGG), the patient having a first biological sex; segmenting the digitized pathology image to identify nuclei; classifying the nuclei as tumor infiltrating lymphocytes (TILs) or non-TILs; extracting sex specific features related to the nuclei identified as the TILs and the non-TILs, the sex specific features characterizing a spatial organization of the TILs and the non-TILs; and operating on the sex specific features with a sex specific regression model corresponding to the first biological sex to generate a sex specific patient risk score.

In yet other embodiments, the present disclosure relates to an apparatus. The apparatus includes a memory configured to store an imaging data set having a digitized pathology image of a patient having or having had high grade glioma (HGG), the patient having a first biological sex; a segmentation tool configured to segment the digitized pathology image to identify nuclei; a classification tool configured to classify the nuclei as tumor infiltrating lymphocytes (TILs) or non-TILs; an extraction tool configured to extract sex specific features related to the nuclei identified as the TILs and the non-TILs, the sex specific features characterizing a spatial organization of the TILs and the non-TILs; and a sex specific machine learning model configured to operate on the sex specific features to generate a sex specific patient risk score.

Examples herein can include subject matter such as an apparatus, including a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
accessing a segmented digitized pathology image corresponding to a high grade glioma (HGG) patient having a first biological sex, wherein the segmented digitized pathology image identifies nuclei;
classifying the nuclei as tumor infiltrating lymphocytes (TILs) or non-TILs;
extracting sex specific features related to the nuclei identified as the TILs and the non-TILs, wherein the sex specific features characterize a spatial organization of the TILs and the non-TILs; and
operating on the sex specific features with a sex specific machine learning model corresponding to the first biological sex to generate a sex specific patient risk score.

2. The method of claim 1, further comprising:
extracting the sex specific features from graphs and nuclei clusters generated from the nuclei identified as the TILs and the non-TILs.

3. The method of claim 1, further comprising:
extracting the sex specific features from the nuclei and from an environment surrounding the nuclei.

4. The method of claim 1, wherein the sex specific features further characterize interactions between the nuclei identified as the TILs and the non-TILs.

5. The method of claim 1, further comprising:
operating the sex specific machine learning model on an additional prognostic attribute including one or more of age and IDH-mutation status.

6. The method of claim 1,
wherein the first biological sex is male; and
wherein the sex specific features are related to one or more of an area of opposing cluster intersection, a graph disorder of non-TILs, a value of TIL density matrix, a non-TIL cluster area, a non-TIL graph triangle area, and a percent of non-TIL clusters surrounding TIL clusters.

7. The method of claim 1,
wherein the first biological sex is female; and
wherein the sex specific features are related to a ratio of an area of opposing cluster intersection to an area of TILs and a percent of non-TIL clusters surrounding other non-TIL clusters.

8. The method of claim 1, wherein the sex specific features further include density features related to the TILs.

9. The method of claim 1, further comprising:
accessing a segmented additional digitized pathology image corresponding to an additional HGG patient having a second biological sex, wherein the segmented additional digitized pathology image identifies additional nuclei;
classifying the additional nuclei as additional TILs or additional non-TILs;
extracting additional sex specific features related to the additional nuclei identified as the additional TILs and the additional non-TILs, wherein the additional sex specific features characterize a spatial organization of the additional TILs and the additional non-TILs; and
operating on the additional sex specific features with an additional sex specific machine learning model corresponding to the second biological sex to generate a second sex specific patient risk score.

10. A non-transitory computer-readable medium storing computer-executable instructions that, when executed, cause a processor to perform operations, comprising:
forming an imaging data set comprising a digitized pathology image of a patient having or having had high grade glioma (HGG), the patient having a first biological sex;
segmenting the digitized pathology image to identify nuclei;
classifying the nuclei as tumor infiltrating lymphocytes (TILs) or non-TILs;
extracting sex specific features related to the nuclei identified as the TILs and the non-TILs, wherein the sex specific features characterize a spatial organization of the TILs and the non-TILs; and
operating on the sex specific features with a sex specific regression model corresponding to the first biological sex to generate a sex specific patient risk score.

11. The non-transitory computer-readable medium of claim 10, wherein the operations further comprise:
separating the digitized pathology image into a plurality of non-overlapping patches; and
identifying the plurality of non-overlapping patches that have more than approximately 50% of HGG tissue; and
segmenting the plurality of non-overlapping patches that have more than approximately 50% of HGG tissue to identify the nuclei, wherein the plurality of non-overlapping patches that are not identified as having more than 50% of the HGG tissue are not segmented.

12. The non-transitory computer-readable medium of claim 10, wherein a support vector machine algorithm is used to classify the nuclei as the TILs and the non-TILs.

13. The non-transitory computer-readable medium of claim 10, wherein the sex specific features are extracted from graphs formed from the nuclei classified as the TILs and the non-TILs and from nuclei clusters formed from the graphs.

14. The non-transitory computer-readable medium of claim 10, wherein the operations further comprise:
constructing graphs separately for TILs and non-TIL using a function that defines connections between nuclei centroids;
adding edges to the graphs using one or more threshold values; and
extracting the sex specific features from the graphs.

15. The non-transitory computer-readable medium of claim 10, wherein the sex specific features further comprise TIL density features.

16. An apparatus, comprising:
a memory configured to store an imaging data set comprising a digitized pathology image of a patient having or having had high grade glioma (HGG), the patient having a first biological sex;
a segmentation tool configured to segment the digitized pathology image to identify nuclei;
a classification tool configured to classify the nuclei as tumor infiltrating lymphocytes (TILs) or non-TILs;
an extraction tool configured to extract sex specific features related to the nuclei identified as the TILs and the non-TILs, wherein the sex specific features characterize a spatial organization of the TILs and the non-TILs; and
a sex specific machine learning model configured to operate on the sex specific features to generate a sex specific patient risk score.

17. The apparatus of claim 16, further comprising:
an additional sex specific machine learning model configured to operate on additional sex specific features to generate an additional sex specific patient risk score; and
wherein the extraction tool is further configured to extract the additional sex specific features from an additional digitized pathology image corresponding to a second biological sex.

18. The apparatus of claim 17, wherein the first biological sex is male and the second biological sex is female.

19. The apparatus of claim 18, wherein the sex specific features are related to one or more of an area of opposing cluster intersection, a graph disorder of non-TILs, a value of TIL density matrix, a non-TIL cluster area, a non-TIL graph triangle area, and a percent of non-TIL clusters surrounding TIL clusters.

20. The apparatus of claim 19, wherein the additional sex specific features are related to the percent of non-TIL clusters surrounding other non-TIL clusters and a ratio of the area of opposing cluster intersection to an area of TILs.
